(19) United States
(12) Patent Application Publication
(10) Pub. No.: US 2009/0123502 A1
(43) Pub. Date: May 14, 2009

(54) MULTI-LAYERED FONDANT CONTAINING CHEWING GUM

(75) Inventors: Steven M. Kumiega, Hummelstown, PA (US); Thomas J. Carroll, Mechanicsburg, PA (US); Todd David Wickenheisser, Harrisburg, PA (US); Mary Ellen Kline, Lancaster, PA (US); Robert J. Huzinec, Hummelstown, PA (US)

Correspondence Address: SCULLY SCOTT MURPHY & PRESSER, PC, 400 GARDEN CITY PLAZA, SUITE 300, GARDEN CITY, NY, 11530, US

(73) Assignee: The Hershey Company, Hershey, PA (US)

(21) Appl. No.: 12/266,861

(22) Filed: Nov. 7, 2008

Related U.S. Application Data
(60) Provisional application No. 61/002,469, filed on Nov. 9, 2007.

(51) Int. Cl. A61K 9/68; A23G 4/18

(52) U.S. Cl. 424/401; 426/5; 424/48

(57) ABSTRACT

The present invention is directed to a multi-layered chewing gum, comprising alternative layers of a chewing gum region comprising a chewing gum composition containing a gum base and a fondant, each layer being separate and discrete and co-extensive, said multilayered chewing gum comprised of at least three layers, wherein the outer layers are comprised of said chewing gum composition, and the fondant layer is sandwiched between the layers comprised of a chewing gum region, said fondant comprised of a first component selected from tastant, a flavor, a sensate and a functional agent and the gum region being comprised of a second component selected from tastant flavor, sensate and a functionality, said first component being distinct from said second component.

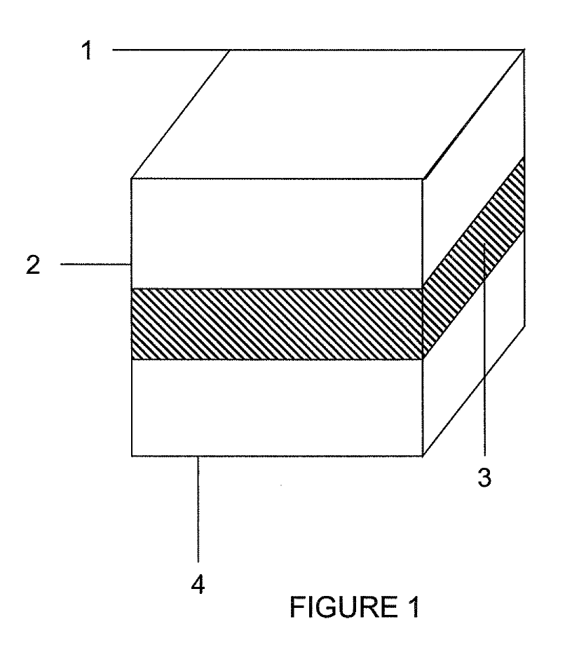

### CROSS-RELATED REFERENCE

This application claims priority of Provisional Application having U.S. Ser. No. 61/002,469, filed on Nov. 9, 2007.

### FIELD OF THE INVENTION

The present invention is directed to a multi-layered chewing gum in which a fondant layer is sandwiched between two layers of a gum composition comprising a gum base.

### BACKGROUND OF THE INVENTION

It is generally known that novel forms of chewing gums are popular with consumers. Over the years, various types of chewing gums products have been introduced. These novel products have utilized various combinations of flavors, sensations, shapes, and texture to interest the consumer. For example, chewing gum products having a dual taste or sensation such as center filled chewing gum products are in popular demand today. Consumers particularly like products that provide a dual flavor perception upon consumption of the product, for it is unlike traditional chewing gum, which merely provides a single gum flavor. Many consumers want more that the single flavor; consumers are desirous of more complex flavored confectionary products.

Furthermore, consumers are always searching for newer and more interesting chewing gum products. Products that can enhance a sensation or taste or allow it to last longer are particularly desirable by consumers. In addition, chewing gums having functional benefits are also of desire.

There is a need, therefore, for new chewing gum compositions, particularly those have a dual taste, flavor or sensations that provide a different and/or longer lasting taste sensation or flavor upon consumption, and for those having functional benefit.

### SUMMARY OF THE PRESENT INVENTION

The present invention is directed to a multi-layered chewing gum, comprising alternate layers of a fondant and a chewing gum region comprising a gum base, each layer being separate and discrete and co-extensive, said multi layered chewing gum comprised of at least three layers, whereas the first and third layers are comprised of the same gum base and the middle layer is comprised of a fondant and is free of any chewing gum base, said fondant layer being sandwiched between the layers comprising the chewing gum region.

In some embodiments, the chewing gum region provides a particular taste and/or sensation or characteristic, and the fondant layer is comprised of the same or complimentary taste and/or sensation or characteristic to reinforce the taste and sensation or characteristic of the chewing gum.

In some embodiments, the chewing gum region provides a first taste and/or sensation and/or characteristic and the fondant layer contains the same or different taste sensation, or characteristics.

In another embodiment, the fondant layer and/or the layer of the chewing gum region imparts functional benefits by providing a functional agent.

### BRIEF DESCRIPTION OF THE DRAWING

The accompanying drawing, which is incorporated and constitutes a part of the present invention, provides an embodiment of the present invention to illustrate the present invention, but the present invention is not limited thereto. The FIGURE represents a perspective view of a three layered chewing gum in which a fondant layer is sandwiched between two layers, each layer comprised of a chewing gum base.

### DETAILED DESCRIPTION

As used herein, the term “ingredient” and the term “component” are used interchangeably to describe any additive, fixing, substance, material, agent, active, element, or part that may be included in the gum compositions of some embodiments.

As used herein, the term “chewing gum” is to include bubble gum, and both are meant to include any gum composition.

As used herein, the term “duality” refers to the presence of two characteristics that are complementary to each other, opposed to each other, i.e., distinct, or different in intensity from each other. The dual characteristics may be flavors, sensations, tastes, functionalities, or other characteristics or benefits. Flavors, sensates, tastants and functional agents also may include compounds that potentate each of these types of components. The dual characteristics also may be color or texture. In some embodiments, the duality may be a dual perception, which refers to the perception by an individual of two characteristics that are complementary to each other, opposed to each other, i.e., distinct, or different in intensity from each other.

The term “multi-modality” refers to the presence of at least two characteristics that are complementary, opposed, i.e., distinct, or different in intensity from one another. The multi-modal characteristics may be flavors, sensations, tastes, functionalities or combinations thereof. Flavors, sensates, tastants and functional agents also may include compounds that potentate each of these types of components. The multi-modal characteristics also could be color or texture. The term “multi-modality” is broader than and encompasses the term “duality” in that it includes embodiments that have a single duality, as well as embodiments that have more than one duality.

The term “complementary” refers to components that are in the same or similar flavor family, for example, the mint family or the fruit family; or components that are in the same or similar sensation family, for example, the cooling family, the warming family or the tingling family; or components that are in the same or similar taste family, for example, the sweetener family, the sour family, the bitter/astringent family, the salty family, the umami family or the kokumi family; or components that are in the same or similar functional family, for example, the breath freshening family. The terms “family” and “type” are used interchangeably herein when referring to multi-modality components.

The term “opposed” means distinctly different components, for example, components that are from different families, such as a component in the flavor family and a component in the taste family.

The term “different in intensity” means that the at least two components that form the duality or multi-modality may be the same component but create the duality or multi-modality by being present in different amounts, by providing a different intensity from one another. This different intensity is formed by the component being in different amounts in one region of the multi-layered chewing gum relative to another region of the multi-layered chewing gum, or from being released at one rate in one region versus being released at another rate in another region of the multi-layered chewing gum.

The present invention is directed to a multi-layered chewing gum, wherein each layer is a discrete and separate layer. The multi layered chewing gum has alternate layers, one layer being comprised of a chewing gum region and the next layer being the fondant layer and then a second chewing gum region, etc. For example, in the accompanying FIGURE, the multilayered chewing gum (1) has an upper outer layer (2) comprising a first chewing gum region, a middle layer comprised of fondant (3), and a lower outer layer comprised of a second chewing gum region (4), which may be the same or different from the first chewing gum region. If the multi-layered chewing has additional layers, the next layer is a fondant layer and the next layer after that is a layer comprised of a third chewing gum region which may be the same as or different from the first or second chewing gum region, and so on. Thus, the multi-layered chewing gum composition has alternate layers of a gum region comprised of a chewing gum base and a layer comprised of a fondant.

The multi-layered chewing gum contains at least three layers, such as depicted in the FIGURE, but it may contain four, five, six or more. It is preferred that it contains less than ten layers and more preferably less than eight layers and even more preferably less than six layers. It is preferred that the outer layers are comprised of the gum base region, so that it is preferred that the multilayered chewing gum of the present invention contains an odd number of layers, e.g., 3, 5, 7 or 9 layers and more preferably, 3 or 5 or 7 layers and even more preferably 3 or 5 layers and most preferably 3 layers.

As shown in the FIGURE, each layer is exposed. Each of the layers in the multi-layered chewing gum has the sides and the ends completely exposed. The outer layers thus do not substantially enclose or encase the middle layers unlike a center-filled chewing gum. However, neither the top nor the bottom of the middle layer is exposed. On the other hand, with respect to the outer layers, in addition to the sides and the ends being exposed, one additional surface is also exposed. With respect to the top layer, the top surface is completely exposed, while the bottom surface thereof completely faces the second layer and is not exposed except at the sides and the ends. With respect to the bottom layer, in addition to the sides and ends, the bottom surface is completely exposed, while the top surface is completely covered by the layer above it.

Moreover, all of the layers are co-extensive, that is, no layer protrudes over another layer or extends past another layer. Thus, all of the layers have the same width and height. However, the various layers may have the same or different thicknesses (depth). Thus, the layers may all have the same volume or different volumes. In one embodiment, none of the layers have the same volume. In another embodiment, at least two of the layers have the same volume, and the other layers are independently thicker, thinner or at the same volume. In another embodiment, two or three of the layers have the same volume and the others are independently thicker or thinner or at the same volume. Thus, for example, in an embodiment, in the three-layered chewing gum of the present invention, two of the layers have the same volume, and the third layer is thicker or thinner. In this embodiment of the three-layered chewing gum, the outer layers (top and bottom) are comprised of the same or different chewing gum regions, while the middle layer is comprised of the fondant, whereby the chewing gum regions have the same volume, and the fondant is thinner. Alternatively, in another embodiment of the three-layered chewing gum, layers comprising the chewing gum regions have the same volume while the fondant layer has a larger volume. In another embodiment of the three-layered chewing gum, all three layers have the same thickness and thus the same volume.

As the number of layers of the multi-layered gum composition exceeds three layers, the number of permutations and combinations of the volumes of each of the layers becomes more and more complex. For example, in the five layered chewing gum, all of the volumes may be different or two of the volumes may be the same (for example, the two outer layers, or one outer layer and one inner layer or the two inner layers have the same volume) or three of the five layers have the same volume or four of the five layers have the same volume or all five layers have the same volume. With respect to the multi-layered chewing gum of the present invention, all combinations and permutations of the volumes and compositions of the various layers are contemplated by the present invention.

It is preferred that the layers having the same type of composition (i.e., the layer having the chewing gum region or the fondant layer) have the same volume. For example, the layers containing the chewing gum region have the same volume, while the fondant may be thicker, thinner or contain the same volume. In another embodiment, all of the layers, i.e., the layer having the chewing gum region and the fondant have the same volume.

In an embodiment of the present invention, the composition of the chewing gum regions contain the same gum base, but may contain the same or different additives therein, for example, the same or different flavor, sensate, taste, color, functional agent, and the like. The fondant, by definition, is different from the chewing gum region in that it has no gum base therein. In an embodiment, all of the chewing gum regions have the same compositions and all of the fondants have the same compositions. In another embodiment, the compositions and the volumes of the chewing gum regions are the same, and all of the fondants have the same composition and volume, although the volume of the chewing gum region and the fondants may be the same or different.

The multi-layered chewing gum can be any suitable size or shape, such as, for example, a pellet, sphere, cube, spiral, cigarette, and the like.

As indicated hereinabove, the multi-layered chewing gum comprises alternate layers comprised of a chewing gum region and a fondant. The layers comprised of the chewing gum region are generally hydrophobic in nature, so that water-soluble components are generally not soluble therein. If the same tastant, sensate flavor, or functional agent is both in the fondant layer and the layer comprised of chewing gum bases, then this difference in solubility would cause the flavor, functional agent, sensation or taste to be released from the fondant layers at a different rate relative to the layer comprised of the chewing gum base.

The chewing gum region in each layer comprises a chewing gum base.

The chewing gum region includes any chewing gum base known in the chewing gum art. For example, the chewing gum base may include elastomers, bulking agents, waxes, elastomer solvents, emulsifiers, plasticizers, fillers or mixtures thereof.

The amount of chewing gum base, which is present in the chewing gum region for each layer, may also vary. In some embodiments, the chewing gum base may be included in the chewing gum region in an amount ranging from about 5% to about 98% by weight of the gum region in each layer. In another embodiment, the amount of chewing gum base ranges from about 15% to about 75% by weight of the chewing gum region of each layer. In another embodiment, the amount of chewing gum base ranges from about 15% to about 50% in weight of the gum region.

Further, the total amount of gum base present in the multi-layer chewing gum composition may vary from one layer comprised of a chewing gum base to another layer comprised of a chewing gum base. In one embodiment, the chewing gum base is present in an amount ranging from about 15% to about 69% by weight of the chewing gum product; in another embodiment, it is present in an amount ranging from about 14% to about 28% of the multi-layered chewing gum and in an embodiment, it is present in an amount ranging from about 25% to about 50% of the chewing gum.

The elastomers (rubbers) employed in the chewing gum base are those typically found in chewing gum bases. Various elastomers can be employed, depending upon various factors, such as the type of chewing gum base desired, the consistency of the chewing gum region desired and the other components used in the chewing gum region to make the final chewing gum product. The elastomer may be any water insoluble polymer known in the art and includes those gum polymers utilized for chewing gums and bubble gums. Illustrative examples of suitable polymers in gum bases include both natural and synthetic elastomers. For example, those polymers which are suitable in gum base compositions include, without limitation, natural substances (of vegetable origin) such as chicle, natural rubber, crown gum, nispero, rosidinha, jelutong, perillo, niger gutta, tunu, balata, guttapercha, lechi capsi, sorva, gutta kay, and the like, and combinations thereof. Examples of synthetic elastomers include, without limitation, styrene-butadiene copolymers (SBR), polyisobutylene, isobutylene-isoprene copolymers, polyethylene, polyvinyl acetate and the like, and combinations thereof.

Additional useful polymers include: crosslinked polyvinyl pyrrolidone, polymethylmethacrylate copolymers of lactic acid, polyhydroxyalkanoates, plasticized ethylcellulose, polyvinyl acetatephthalate and combinations thereof:

The amount of elastomer employed in the gum base in each layer may vary depending upon various factors such as the type of gum base used, the consistency of the gum composition desired and the other components used in the composition to make the final chewing gum product. In general, the elastomer will be present in the gum base in each layer in an amount from about 5% to about 20% by weight of the gum region in each layer, desirably from about 7% to about 20% by weight of each gum region layer.

In some embodiments, the gum base may additionally include a wax. It softens the polymeric elastomer mixture and improves the elasticity of the gum base. When present, the waxes employed will have the melting points of waxes typically found in chewing gum. They may be high or low melting waxes. The low melting wax may be a paraffin wax. The wax may be present in the gum base in each layer in an amount ranging from about 0% to about 20%, and preferably from about 5% to about 15%, by weight.

In addition to the low melting point waxes, waxes having a higher melting point may be used in the gum base in amounts from about 0% up to about 10% by weight of the gum base in each layer. Such high melting waxes include beeswax, vegetable wax, candelilla wax, carnuba wax, most petroleum waxes, and the like, and mixtures thereof.

Examples of waxes that may be present in the gum base in each layer includes waxes, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, mixtures thereof, and the like.

In addition to the components set out above, the gum base may include a variety of other ingredients, such as components selected from elastomer solvents, emulsifiers, plasticizers, fillers, and mixtures thereof.

The gum base may contain elastomer solvents to aid in softening the elastomer component. Such elastomer solvents may include those elastomer solvents known in the art, for example, terpinene resins such as polymers of alpha-pinene or beta-pinene, methyl, glycerol and pentaerythritol esters of rosins and modified rosins and gums such as hydrogenated, dimerized and polymerized rosins, and mixtures thereof. Examples of elastomer solvents suitable for use herein may include the pentaerythritol ester of partially hydrogenated wood and gum rosin, the pentaerythritol ester of wood and gum rosin, the glycerol ester of wood rosin, the glycerol ester of partially dimerized wood and gum rosin, the glycerol ester of polymerized wood and gum rosin, the glycerol ester of tall oil rosin, the glycerol ester of wood and gum rosin and the partially hydrogenated wood and gum rosin and the partially hydrogenated methyl ester of wood and rosin, and the like, and mixtures thereof. The elastomer solvent may be employed in the gum base in each layer in amounts from about 0% to about 30%, and preferably from about 5% to about 15%, by weight.

The gum base may also include emulsifiers, which aid in dispersing the immiscible components into a single stable system. The emulsifiers useful in this invention include glyceryl monostearate, lecithin, fatty acid monoglycerides, diglycerides, propylene glycol monostearate, and the like, and mixtures thereof. The emulsifier may be employed in amounts from about 0.1% to about 10%, and more specifically, from about 3% to about 10%, by weight of the gum base in each layer.

The gum base may also include plasticizers or softeners to provide. a variety of desirable textures and consistency properties. Because of the low molecular weight of these ingredients, the plasticizers and softeners are able to penetrate the fundamental structure of the gum base, making it plastic and less viscous. Useful plasticizers and softeners include lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine, and the like, and mixtures thereof. The plasticizers and softeners are generally employed in the gum base in each layer in amounts up to about 20% by weight of the gum base in each layer, and more specifically in amounts from about 4% to about 15%, by weight of the gum base in each layer.

The plasticizers in the gum base of each layer may include hydrogenated vegetable oils, such as soybean oil and cottonseed oil, which may be employed alone or in combination. These plasticizers provide the gum base with good texture and soft chew characteristics. These plasticizers and softeners are preferably employed in amounts from about 0.1% to about 20%, and more specifically in amounts from about 5% to about 14, by weight of the gum base in each layer.

Glycerin, which may be either anhydrous or hydrous, may also be employed as a softening agent, such as the commercially available United States Pharmacopeia (USP) grade. Glycerin is a syrupy liquid with a sweet warm taste and has a sweetness of about 60% of that of cane sugar. Because glycerin is hygroscopic, anhydrous glycerin may be maintained under anhydrous conditions throughout the preparation of the chewing gum composition.

In some embodiments, the gum base in each layer may also include effective amounts of bulking agents such as mineral adjuvants, which may serve as fillers, and textural agents. Useful mineral adjuvants include calcium carbonate, magnesium carbonate, alumina, aluminum hydroxide, aluminum silicate, talc, tricalcium phosphate, dicalcium phosphate, calcium sulfate and the like, and mixtures thereof. These fillers or adjuvants may be used in the gum base compositions in various amounts. The bulking agents is preferably present in an amount ranging from about 0.1 to about 60%, and more preferably from about 0.1% to about 40%, by weight of the gum base in each layer. In some embodiments, the amount of bulking agent is from about 0.1 to about 30% by weight.

A variety of traditional ingredients may also be optionally included in the gum base in effective amounts such as coloring agents, antioxidants, preservatives, flavoring agents, high intensity sweeteners, and the like. For example, titanium dioxide and other dyes suitable for food, drug and cosmetic applications, known as F. D. & C. dyes, may be utilized. An anti-oxidant such as butylated hydroxytoluene (BHT), butylated hydroxyanisole (BHA), propyl gallate, and mixtures thereof, may also be included. Other conventional chewing gum additives known to one having ordinary skill in the chewing gum art may also be used in the gum base.

The chewing gum region may include conventional additives selected from the group consisting of sweetening agents (sweeteners), additional plasticizers, additional softeners, additional emulsifiers, additional waxes, additional fillers, bulking agents (carriers, extenders, bulk sweeteners), mineral adjuvants, flavoring agents (flavors, flavorings), additional coloring agents (colorants, colorings), antioxidants, acidulants, thickeners, and the like, and mixtures thereof. Some of these additives may serve more than one purpose. For example, in sugarless gum compositions, a sweetener, such as maltitol or other sugar alcohol, may also function as a bulking agent.

The plasticizers, softening agents, mineral adjuvants, waxes and antioxidants discussed above, as being suitable for use in the gum base, may also be used in the chewing gum region. Examples of other conventional additives which may be used include emulsifiers, such as lecithin and glyceryl monostearate, thickeners, used alone or in combination with other softeners, such as methyl cellulose, alginates, carrageenan, xanthan gum, gelatin, carob, tragacanth, locust bean gum, pectin, alginates, galactomannans such as guar gum, carob bean gum, glucomannan, gelatin, starch, starch derivatives, dextrins and cellulose derivatives such as carboxy methyl cellulose, acidulants such as malic acid, adipic acid, citric acid, tartaric acid, fumaric acid, and mixtures thereof, and fillers, such as those discussed above.

In some embodiments, the chewing gum region may also contain a bulking agent. Suitable bulking agents may be water-soluble and include sweetening agents selected from, but not limited to, monosaccharides, disaccharides, polysaccharides, sugar alcohols, and mixtures thereof; randomly bonded glucose polymers such as those polymers distributed under the tradename Litesse™ which is the brand name for polydextrose and is manufactured by Danisco Sweeteners, Ltd. of 41-51 Brighton Road, Redhill, Surryey, RH1 6YS, United Kingdom.; isomalt (a racemic mixture of alpha-D-glucopyranosyl-1,6-mannitol and alpha-D-glucopyranosyl-1,6-sorbitol manufactured under the tradename PALATINIT by Palatinit Sussungsmittel GmbH of Gotlieb-Daimler-Strause 12 a, 68165 Mannheim, Germany; maltodextrins; hydrogenated starch hydrolysates; hydrogenated hexoses; hydrogenated disaccharides; minerals, such as calcium carbonate, talc, titanium dioxide, dicalcium phosphate; celluloses; and mixtures thereof.

Suitable sugar bulking agents include monosaccharides, disaccharides and polysaccharides such as xylose, ribulose, glucose (dextrose), lactose, mannose, galactose, fructose (levulose), sucrose (sugar), maltose, invert sugar, partially hydrolyzed starch and corn syrup solids, and mixtures thereof.

Suitable sugar alcohol bulking agents include sorbitol, xylitol, mannitol, galactitol, lactitol, maltitol, erythritol, isomalt and mixtures thereof.

Suitable hydrogenated starch hydrolysates include those disclosed in U.S. Pat. No. 4,279,931 and various hydrogenated glucose syrups and/or powders which contain sorbitol, maltitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN®, a commercially available product manufactured by Roquette Freres of France, and HYSTAR®, a commercially available product manufactured by SPI Polyols, Inc. of New Castle, Del., are also useful.

The sweetening agents, which may be included in the chewing gum regions embodiments, may be any of a variety of sweeteners known in the art. These may be used in many distinct physical forms well-known in the art to provide an initial burst of sweetness and/or a prolonged sensation of sweetness. Without being limited thereto, such physical forms include free forms, such as spray dried, powdered, beaded forms, encapsulated forms, and mixtures thereof.

The intense sweetener is a high intensity sweetener. Examples of high intensity sweeteners as aspartame, neotame, sucralose, and acesulfame potassium (Ace-K), stevoside and the like.

In general, an effective amount of intense sweetener may be utilized to provide the level of sweetness desired, and this amount may vary with the sweetener selected. In some embodiments the amount of intense sweetener may be present in amounts from about 0.001% to about 3%, by weight of the gum region of each layer, depending upon the sweetener or combination of sweeteners used. The exact range of amounts for each type of sweetener is easily determined by those skilled in the art, depending upon the sweetness intensity desired.

The gum region also contains a bulk sweetener. The bulk sweetener may be a solid sugar or polyol. The sugar may be a natural sugar. Sugar sweeteners generally may include saccharide-containing components commonly known in the confectionery art, including but not limited to sucrose, dextrose, maltose, dextrin, dried invert sugar, fructose, levulose, tagatose, galactose, corn syrup solids, and the like, alone or in combination. Alternatively, sweeteners include glycerin, fruit concentration and fruit pastes.

Alternatively, or concurrently, the bulk sweetener may be a polyol. The polyol may include any polyol known in the art that impart sweetness. The polyol may include maltitol, sorbitol, erythritol, xylitol, mannitol, isomalt, lactitol and combinations, thereof. Lycasin™ which is a hydrogenated starch hydrolysate, including sorbitol and maltitol may be used. Blends of two or more different polyols may also be used. Examples of polyols which can be used as blends include xylitol, maltitol, sorbitol, lactitol, mannitol, isomalt or combinations thereof.

Since sugar (sucrose) is generally accepted as the baseline for sweetness intensity comparison of sweeteners, including polyols, the polyol composition of some embodiments is described similarly. For example, the polyol composition of may have a sweetness of greater than about 50% of the sweetness of sucrose. More specifically, the polyol composition of the present invention may have sweetness greater than about 70% of the sweetness of sucrose.

These sugar and polyol and sweeteners are present in amounts sufficient to impart a desired sweetness to the chewing region of the gum. The amount of sweetener present is dependent upon whether the sweetener is a sugar, polyol or combination. However, in another embodiment, the sweetener is a polyol or combination of polyols.

The bulk sweeteners are present in the chewing gum region in one embodiment ranging from about 40% to about 75% by weight and in another embodiment, from about 45% to about 65% by weight.

If the chewing gum is a sugar containing gum, then the sweetness in each of the chewing gum regarding each layer may independently be a sugar polyol or high potency sweetener or combination thereof. However, if a sugarless chewing gum is the product, then the sweetener cannot be a sugar, but rather is independently polyol or combination of polyols and optionally, in addition a high potency sweetener or combination of high potency sweetener.

The amount of and type of the bulk sweetener used in the gum region will depend on many factors including the type of elastomers used in the gum base and the particular polyols used. If the gum contains sugar, then the bulk sweetener is sugar, such as those described hereinabove or a polyol or combination of polyol or a combination of one or more polyols and one or more sugars. If the gum is a sugarless gum, then the bulk sweetener is a polyol or a combination of polyols.

Coloring agents in the gum region may be used in amounts effective to produce the desired color. The coloring agents may include pigments which may be incorporated in amounts from about 0% up to about 6%, by weight of the gum composition. For example, titanium dioxide may be incorporated in amounts up to about 2%, and preferably less than about 1%, by weight of the gum composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-p-sulfoniumbenzylamino) diphenylmethylene]-[1-(N-ethyl-N-p-sulfoniumbenzyl)-delta-2,5-cyclohexadieneimine]. A full recitation of all F.D.& C. colorants and their corresponding chemical structures may be found in the Kirk-Othmer Encyclopedia of Chemical Technology, 3rd Edition, in volume 5 at pages 857-884, which text is incorporated herein by reference.

Suitable oils and fats usable in gum compositions include partially hydrogenated vegetable or animal fats, such as coconut oil, palm kernel oil, beef tallow, and lard, among others. These ingredients when used are generally present in amounts up to about 7%, and preferably up to about 3.5%, by weight of the chewing gum region in each layer.

The gum region may have a total moisture content of less than about 10% by weight of the gum region and more specifically may have a total moisture content from about 0.1% to about 6.0% by weight.

The fondant layer is the other layer. The fondant layer is comprised of a bulk sweetener and water which has been cooked to a point, specifically the soft ball stage, then cooled slightly and then stirred or beaten until it is an opaque mass of creamy consistency. It contains no gum base.

The bulk sweetener provides the sweetness. It may be a solid sugar or polyol commonly used in fondants. If it is a sugar, then the sugar may be a natural sugar. Sugar sweeteners generally may include saccharide-containing components commonly known in the confectionery art, including but not limited to sucrose, dextrose, maltose, dextrin, dried invert sugar, fructose, levulose, tagatose, galactose, corn syrup solids, and the like, alone or in combination. Alternatively, sweeteners include glycerin, fruit concentration and fruit pastes.

Alternatively, or concurrently, the fondant may include a polyol. The polyol may include any polyol known in the art that impart sweetness. The polyol may include maltitol, sorbitol, erythritol, xylitol, mannitol, isomalt, lactitol and combinations, thereof. Lycasin™ which is a hydrogenated starch hydrolysate, including sorbitol and maltitol may be used. Blends of two or more different polyols may also be used. Examples of polyols which can be used as blends include xylitol, maltitol, sorbitol, lactitol, mannitol, isomalt or combinations thereof.

The sweetener is present in amounts sufficient to impart a desired sweetness to the fondant layer of the gum. The amount of sweetener present is dependent upon whether the sweetener is a sugar, polyol or combination. If the sweetener is a sugar, in an embodiment, it may comprise up to about 98% of polyol of the fondant layer by weight and in another embodiment from about 90% to 98% by weight and in another embodiment from about 85% by weight to about 98% by weight. However, in another embodiment, the sweetener is a polyol or combination of polyols. In such situation, the polyol or combination of polyol is present in amounts ranging from about 80% to about 95% by weight the fondant and in another embodiment from about 60% to about 90%. For example, if the fondant contains a combination of polyols, such as xylitol and Lycasin™, one polyol, e.g. xylitol may be present in amounts ranging from about 50% to about 90% by weight and in another embodiment from about 60% to about 80% by weight and in another embodiment, from about 65% to about 75% by weight (e.g. 70%), while the other polyol, e.g., Lycasin™, may be present in about 10% to about 30%, and in another embodiment from about 15% to about 25% by weight of the fondant layer.

The total bulk sweetener is present in the fondant layer in one embodiment in an amount ranging from about 80 to about 99% by weight and in another embodiment, from about 85 to about 95% by weight. The bulk sweetener component in an embodiment does not exceed about 95% by weight of the fondant layer.

As with the gum layer, the type of sweetener and the amount of sweetener independent upon several factors, such as the type of product (sugar or sugarless), the compensation of the fondant, and the like. If the multi-layered chewing gum contains sugar, then the bulk sweetener is a polyol or a solid sugar, such as the ones described hereinabove. Alternatively, if the multi-layered chewing gum is a sugarless gum, no sugar is present, then the bulk sweetener is one or more polyols.

The fondant layer may also optionally contain high intensity sweeteners, such as those described hereinabove in the gun region layer, the contents of which are incorporated by reference. If present, they are present in amounts ranging from about 0.0001% to about 3% by weight of the fondant.

The fondant layer is a hydrophilic layer. It contains from about 3% to about 15% by weight of water in one embodiment, while in another embodiment, about 5% to about 10%, by weight water and in another embodiment from about 6% to about 8% by weight water. The water activity of the fondant layer ranges from about 0.50 to about 0.70 and more preferably from about 0.55 to about 0.65.

The water activity of the fondant layer is greater than the water activity of the gum region. As indicated hereinabove, the water activity of the fondant layer ranges from about 0.55 to about 0.65, while in the gum region, the water activity ranges from about 0.40 to about 0.50.

Other components may be present in the fondant layer. For example, the fondant layer optionally contains a flavorant. The flavorant is any flavorant or flavor used in the confectionary art. It may be a natural or synthetic oil and/or flavor as is commonly known in the art. Natural and artificial flavoring agents may be combined in any sensorially acceptable fashion. The flavor agents can be used in any suitable amounts.

Non limiting examples of suitable flavorants include natural and synthetic flavoring agents chosen from synthetic flavor oils and flavoring aromatics and/or oils, oleo resins, and extracts derived from plants, leaves, flowers, fruits, vegetables and the like and/or combinations therein. Non-limiting examples of flavor oils include spearmint oil, cinnamon oil, oil of wintergreen (methyl salicylate), peppermint oils, clove oils, bayoil arise oil, eucalyptus oil, thyme oil, cedar leaf oil, oil of nutmeg, oil of sage, oil of bitter almonds, cassin oil and the like. Alternatively, fruit flavors may be present, which may be artificial, natural or synthetic. Examples include vanilla cream, caramel, banana, cocoa, citrus oil, such as lemon, orange, grape, lime, grapefruit, and fruit essences, including apple, pear, peach, strawberry, raspberry, cherry, plum, pineapple, apricot and the like. Flavors may also include pastes, capsules, beads, films, crystals, powders, and extracts of fruits and vegetables.

The fondant may also contain chocolate flavor, i.e., the fondant layer is a chocolate fondant. In addition to the sweeteners described hereinabove, it contains the ingredients typically found in chocolate fondants. They include cocoa butter, cocoa powder, cocoa liquor, cocoa solids, milk fat and non-milk fat solids.

Polysaccharides, such as Ticalose 15 Pre-hydrated (carboxy methylcellulose) or hydrophilic sustained release gums may additionally be present. For examples, the polysaccharides may interact with the polyol present to provide the proper texture. The polysaccharide is present in an amount sufficient so that together with polyol, such as maltitol, the fondant layer has the proper malleability so that it will not crack during the manufacturing processing, especially in the cooling tunnel as described hereinbelow. In an embodiment, it is present in amounts ranging from about 0.1 to about 3% by weight of the fondant and more preferably from about 0.40 to about 2% by weight of the fondant and in another embodiment from about 0.50 to about 1.5% by weight the fondant.

The fondant may also include coloring agents in amounts effective to produce the desired color. The coloring agents may include pigments, which may be incorporated in amounts up to about 1% by weight of the fondant. Examples include titanium dioxide, natural food colors and dyes suitable for food, drug and cosmetic applications, such as the FD&C dyes and the like.

Other conventional additives may be additionally be present in the fondant, such as softeners, mineral adjuvants, antioxidants, anti-microbials, acidulants, and mixtures thereof.

The present invention imparts a flavor duality, a sensitive duality, a taste duality to the chewing gum, or it may incorporate functional agents therein. In creating these dualities, complementary or opposed components that differ in intensity are included in gum region layers and/or in the fondant layers. These may affect the flavor, the sensation, or taste. In another embodiment, the functional features, such as the drug, dental whitener and the like, are present in the fondant layer or in the gum region or both.

### Flavor

Some embodiments provided herein may extend to combinations that include a duality. For example, one component to create a duality may be present in the fondant and another in the gum region. For instance, a first flavor may be present in the gum region and the second flavor may be present in the fondant. The flavors may be distinct from one another, complementary to one another or different in intensities from one another. Two or more components may be present in the multi-layered chewing gum of the present invention. For example, if three components are present, one component may be present in the gum region and two in the fondant layer. For example, the fondant may contain more than one flavor component, which may be opposed complementary to the flavor component present in the gum region or flavor components in the fondant or it may be different in intensity to the other flavor components in the gum region or the gum region may contain more than one flavor component which may be opposed, or complementary to the flavor component in the fondant layer or any other flavor in the gum region or it may be different in intensity to the flavor components in the fondant layer or flavors in the gum regions. In another embodiment, the flavor components may be present in both the fondant layer and the gum region, whereby the flavor components in the gum region is complementary to, opposed to or different in intensity to the flavors that are present in the fondant layer.

However, in a preferred embodiment, a first flavor is present in the gum region and the second flavor is present in the fondant region. The second flavor may be distinct from, complementary to or different in intensity from the first flavor.

In some embodiments, the fondant may include the first flavor, the gum region the second and possible a third flavor wherein each gum region has a different flavor or the same flavor. In such embodiments, the fondant flavor may be distinct from, complementary to or different in intensity from the flavor in the various gum regions. In other embodiments, one of the gum region flavors may be complementary to or opposed to the flavor in the fondant or differ in intensity from the other flavor in the gum region of another layer in the multi-layered chewing gum, while being complementary to, opposed to or differ in intensity to the fondant flavor. For example, the gum regions may be two different mint flavors, such as, peppermint and spearmint. The fondant may be distinct from the mint flavors, such as, for example, cinnamon.

A variety of flavors may be used to impart different dualities. More specifically, in some embodiments, at least two flavors that are distinct may be employed. Dualities based on distinct flavors may include, but are not limited to, the following combinations: a mint flavor and a fruit flavor; a mint flavor and a spicy flavor; a mint flavor and a savory flavor; a mint flavor and an indulgent flavor; a fruit flavor and a spicy flavor; a fruit flavor and a savory flavor; a fruit flavor and an indulgent flavor; a spicy flavor and a savory flavor; a spicy flavor and an indulgent flavor; and a savory flavor and an indulgent flavor.

Some of the duality combinations set forth above include an indulgent flavor. As used herein, “indulgent” refers to a type of flavor associated with a creamy or decadent taste. Sometimes these flavors are referred to as “sweet/brown” in the art. Examples of suitable indulgent flavors include, but are not limited to, maple, cola, chocolate, dulce de leche, raisin, vanilla, caramel, dairy flavors, such as cream, butter, milk and yogurt, butterscotch, peanut butter, fruit cream flavors, such as strawberry cream, and combinations thereof.

In some embodiments, at least two flavors that are complementary may be employed. In some embodiments, the complementary flavors may be the same type of flavor, e.g., two different mint flavors. In some other embodiments, a first flavor, e.g., a fruit flavor, may be provided, and the second flavor may be complementary by enhancing the first flavor, e.g., a fruit potentiator. More specifically, dualities based on complementary flavors may include, but are not limited to, the following combinations: a mint flavor and a mint potentiator; a fruit flavor and a fruit potentiator; a spicy flavor and a spice potentiator; a savory flavor and a savory potentiator; a mint flavor and a different mint flavor; a fruit flavor and a different fruit flavor; a spicy flavor and a different spicy flavor; a savory flavor and a different savory flavor; and an indulgent flavor and a different indulgent flavor.

In some embodiments, the duality may be based on at least two portions of a flavor that differ in intensity. For instance, any of the following types of flavors may be used in at least two portions, each of which contains a different amount of the flavor: mint flavor; fruit flavor; spicy flavor such as spicy chili powder; savory flavor; and indulgent flavor. For example, one of the regions of the chewing gum may include a first amount of a flavor and a separate region may include a second amount of the same flavor. The second amount may be greater than the first amount of the flavor or vice versa, thereby creating an intensity differential in the flavor impact.

A variety of exemplary flavors, include mint, fruit, spicy, savory and indulgent flavors.

### Sensate

With respect to sensate dualities, some gum compositions of the present invention may include a duality based on sensations, such as coolness, warmth, salivating and tingling sensations. Such sensations may be provided by sensates, such as cooling agents, warming agents or tingling agents, respectively. In some embodiments, different sensates are present in the gum region and in the fondant. The second sensate may be distinct from, complementary to or different in intensity from the first sensate. For instance, the fondant region may include the first sensate and the gum region may include the second sensate.

A variety of sensates may be used to impart different dualities. More specifically, in some embodiments, at least two sensates that are distinct may be employed. Dualities based on distinct sensates may include, but are not limited to, the following combinations: a cooling agent and a warming agent; a cooling agent and a tingling agent; and a warming agent and a tingling agent, a salivating agent and a tingling agent, a salivating agent and a warming agent or a cooling agent.

In some embodiments, at least two sensates that are complementary may be employed. In particular, the complementary sensates may be the same type of sensate, such as, two different cooling agents, two different warming agents or two different tingling agents or two different salivating agents.

In some embodiments, the duality may be based on at least two portions of a sensate that differ in intensity. Any of the following types of sensates may be used in the gum region or the fondant portions, each of which contains a different amount of the sensate: cooling agents, warming agents, salivating agents or tingling agents. For example, the gum region of the chewing gum may include a first amount of a sensate and the fondant may include a second amount of the same sensate. The first amount may be greater than the second amount of the sensate, thereby creating an intensity differential in the sensation.

It is known to those skilled in the art that by blending sensates, early and extended perceptions are affected.

To enhance the perception of both flavored sensates, such as cooling, the flavored sensates can be added separately to the fondant and to the gum region layers. The differences in dissolution of the sensates in the fondant relative to the gum region will further enhance the desired effect. For example, flavored menthol may be added to the gum region of the layer or to the fondant. In another embodiment, cooling sensates are added to the gum regions and/or fondant. In a third embodiment, flavor and Cooling sensates can be present in the gum and/or fondant. In another embodiment, addition of flavor, menthol and cooling sensates can be present in the gum region and/or fondant.

For example, in an embodiment WS3 (N-2,3-trimethyl-2-isopropylbutamide, a cooling sensate, and flavor is found in the gum region layer and menthol and flavors in the fondant layer. In another embodiment, WS23 (2-isopropyl-N-2,3-trimethyl-butyramide), a cooling sensate, and flavor are present in the gum region layers, while menthol and flavor are present in the fondant layer. Alternatively, WS3 and flavor are present in the fondant layer and menthol and flavor are present in the gum region layers. In another embodiment, WS23 and flavor are found in the fondant and menthol and flavor is in the gum region layers. In-a still further embodiment, WS3 and WS 23, and flavor are present in the gum region layers and menthol and flavor are present in the fondant. In a still further embodiment, WS3 and WS 23 are in the fondant layer and menthol and flavor in the gum region layers.

### Taste

With respect to taste dualities, some gums may include a duality based on tastes, such as, bitter, salty, sweet, sour, umami and kokumi tastes. Tastants are agents that may provide such tastes. In some embodiments, the gum region may include a first tastant and the fondant includes at least a second tastant. The second tastant may be distinct from, complementary to or different in intensity from the first tastant.

A variety of tastants may be used to impart different dualities. More specifically, in some embodiments, at least two tastants that are distinct may be employed. Dualities based on distinct tastes may include, but are not limited to, the following combinations: a sweet tastant and a sour tastant; a sweet tastant and a salty tastant; a sweet tastant and a bitter tastant; a sweet tastant and an astringent tastant; a sweet tastant and an umami tastant; a sweet tastant and a kokumi tastant; a sour tastant and a salty tastant; a sour tastant and a bitter tastant; a sour tastant and an astringent tastant; a sour tastant and an umami tastant; a sour tastant and a kokumi tastant; a salty tastant and a bitter tastant; a salty tastant and an astringent tastant; a salty tastant and an umami tastant; a salty tastant and a kokumi tastant; a bitter tastant and an astringent tastant; a bitter tastant and an umami tastant; and a bitter tastant and a kokumi tastant.

In some embodiments, at least two tastants that are complementary may be employed. In particular, the complementary tastants may be the same type of tastant, such as, two different bitter agents; two different sour agents, two different sweeteners; two different salts; two different umami agents; or two different kokumi agents.

In some embodiments, the duality may be based on at least two portions of a tastant that differ in intensity. Any of the following types of tastants may be used in at least two portions, each of which contains a different amount of the tastant: bitter agents; sour agents, sweeteners; salts; umami agents; or kokumi agents. For example, the fondant region of the chewing gum may include a first amount of a tastant and a gum region layer may include a second amount of the same tastant. The second amount may be greater than the first amount of the tastant, thereby creating an intensity differential in the taste, especially in odd number of multilayers.

Some of the duality combinations set forth above include an umami tastant. “Umami” refers to a taste that is savory, or the taste of glutamate.

### Functional Agents

With respect to functional agents, they may be present in either one or the other or both the fondant and the gum region. If both the fondant and the gum region contain functional agents, they may be complementary or differ in intensity or they may be two different functional agents that perform the same function. The functionalities include, for example, teeth whitening and breath freshening, among others, and may be provided by various functional agents. The second functional agent may be distinct from, complementary to or different in intensity from the first functional agent. For instance, the fondant may include a first functional agent and the gum region may include a second functional agent.

The functional agents in the gum region and fondant may be two different anti-plaque agents, such as, chlorhexidine and triclosan. In another embodiment, the gum region area may have one drug, such as anti-plaque agent, while the fondant layer may contain a functional agent that is distinct from the anti-plaque agents, such as, for example, a remineralization agent.

A variety of functional agents may be used to impart different dualities. More specifically, in some embodiments one or two functional agents that are distinct may be employed. The functional agents may include, but are not limited to: a vitamin, a mineral or combination thereof; a breath freshening agent and a tooth whitening agent or combination thereof, a breath freshening agent and a remineralization agent or combination thereof; a breath freshening agent and an antimicrobial agent or combination thereof; a tooth whitening agent and a stain prevention agent or combination thereof; an appetite suppressant and a stress relieving agent or combination thereof, an energy boosting agent and a stress relieving agent or combination thereof; a concentration enhancing agent and a focus enhancing agent or combination thereof, and the like.

In some embodiments, two functional agents that are complementary may be employed. In particular, the complementary functional agents may be the same type of functional agent, such as, two different surfactants, two different breath freshening agents, two different anti-microbial agents, two different antibacterial agents, two different anti-calculus agents, two different anti-plaque agents, two different fluoride compounds, two different quaternary ammonium compounds, two different remineralization agents, two different pharmaceutical actives, two different micronutrients, two different throat care actives, two different tooth whitening agents, two different stain removing agents, two different energy boosting agents, two different concentration boosting agents, two different flavor enhancing agents and two different appetite suppressants and the like.

In some embodiments, the duality may be based on at least two portions of a functional agent that differ in intensity. Any of the types of functional agents set forth above in the description of complementary functional agents may be used in at least two portions, each of which contains a different amount of the functional agent. For example, the gum region of the chewing gum may include a first amount of a functional agent and a separate region may include a second amount of the same functional agent. The second amount may be greater than the first amount of the functional agent, thereby creating an intensity differential in the functionality. Alternatively, the functionality may be present in either one of the fondant layer or the gum region. In one embodiment, it is preferred to be present in at least the fondant layer. If the functional agents are the same in both the fondant layer and the gum region layer, then the solubility in the fondant layer and the gum region layer of the chewing gum are different, thus, the functional agent will be released at different rates from the two regions of the gum. As a result, the effect of the functional agent will be prolonged.

Alternatively, the gum region may contain one category selected from flavors, sensates, tastants and functional agents, while the fondant may contain a component selected from different category of sensates, testants, flavors and functional agents. In another embodiment, the fondant may contain any one, two, three, four components from different aforementioned categories of sensates, tastants, flavors and functional agents, and the gum region may contain any one, two, three or four components from a different category. All combinations and permutations are contemplated. In another embodiment, the gum region may contain one or more of the categories of flavors, tastants, sensates or functional agents, where by the categories selected for the fondant and the gum region may be the same or different. Any combination/or permutation thereof is contemplated by the present invention.

It is to be noted that since the gum region and the fondant are comprised of different ingredients, they both have different textures, which is sensed by the consumer when biting into the chewing gum of the present invention. Thus, the present invention imparts different texture feelings to the consumer.

### Sweeteners

The sweeteners involved may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:

(a) water-soluble sweetening agents such as dihydrochalcones, monellin, steviosides, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame), N—[N-(3,3-dimethylbutyl)-L-.alpha.-aspartyl]-L-phenylalanine 1-methyl ester Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fiuctofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro 1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro-4,6,1′,6′-tetradeoxygalacto-sucrose; and 4,6,1′,6′-tetradeoxy-sucrose, and mixtures thereof;

(e) protein based sweeteners such as thaumaoccous danielli (Thaumatin I and II) and talin; and

(f) the sweetener monatin (2-hydroxy-2-(indol-3-ylmethyl)-4-aminoglutaric acid) and its derivatives.

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K).

The active component (e.g., sweetener), which is part of the delivery system, may be used in amounts necessary to impart the desired effect associated with use of the active component (e.g., sweetness). In general, an effective amount of intense sweetener may be utilized to provide the level of sweetness desired, and this amount may vary with the sweetener selected. The intense sweetener may be present in amounts from about 0.001% to about 3%, by weight of the composition, depending upon the sweetener or combination of sweeteners used. The exact range of amounts for each type of sweetener may be selected by those skilled in the art.

In another embodiment, the fondant may be comprised of chocolate, i.e., a chocolate. The chocolate fondant center is prepared in accordance with conventional techniques known in the art.

### Sensate Ingredients

Sensate compounds can include cooling agents, warming agents, tingling agents, effervescent agents, and combinations thereof A variety of well known cooling agents may be employed. For example, among the useful cooling agents are included xylitol, erythritol, dextrose, sorbitol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N,2,3-trimethyl-2-isopropyl butanamide (WS-23), N-ethyl-p-menthane-3-carboxamide (WS-3), isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide, n-ethyl-t-2-c-6 nonadienamide, N,N-dimethyl menthyl succinamide, substituted p-menthanes, substituted p-menthane-carboxamides, 2-isopropanyl-5-methylcyclohexanol (from Hisamitsu Pharmaceuticals, hereinafter “isopregol”); menthone glycerol ketals (FEMA 3807, tradename FRESCOLAT® type MGA); 3-1-menthoxypropane-1,2-diol (from Takasago, FEMA 3784); and menthyl lactate; (from Symrise, FEMA 3748, tradename FRESCOLAT® type ML), WS-30, WS-14, Eucalyptus extract (p-Mehtha-3,8-Diol), Menthol (its natural or synthetic derivatives), Menthol PG carbonate, Menthol EG carbonate, Menthol glyceryl ether, N-tertbutyl-p-menthane-3-carboxamide, P-menthane-3-carboxylic acid glycerol ester, Methyl-2-isopropyl-bicyclo(2.2.1), Heptane-2-carboxamide; and Menthol methyl ether, and menthyl pyrrolidone carboxylate among others. These and other suitable cooling agents are further described in the following U.S. patents, all of which are incorporated in their entirety by reference hereto: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago International Corporation (USA) Rockleigh, N.J., vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation can be provided. One such tingling sensation is provided by adding jambu, oleoresin, or spilanthol to some examples. In some embodiments, alkylamides extracted from materials such as jambu or sanshool can be included. Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of “tingling” type sensates can be found in U.S. Pat. No. 6,780,443, the entire contents of which are incorporated herein by reference for all purposes.

Sensate components may also be referred to as “trigeminal stimulants” such as those disclosed in U.S. Patent Application No. 205/0202118, which is incorporated herein by reference. Trigeminal stimulants are defined as an orally consumed product or agent that stimulates the trigeminal nerve. Examples of cooling agents which are trigeminal stimulants include menthol, WS-3, N-substituted p-menthane carboxamide, acyclic carboxamides including WS-23, methyl succinate, menthone glycerol ketals, bulk sweeteners such as xylitol, erythritol, dextrose, and sorbitol, and combinations thereof. Trigeminal stimulants can also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin and menthol glycerin ether, unsaturated amides, and combinations thereof.

### Breath Freshening Ingredients

Breath fresheners can include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments breath fresheners can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof. In some embodiments, the release profiles of probiotics can be managed for a gum including, but not limited to lactic acid producing microorganisms such as Bacillus coagulans, Bacillus subtilis, Bacillus laterosporus, Bacillus laevolacticus, Sporolactobacillus inulinus, Lactobacillus acidophilus, Lactobacillus curvatus, Lactobacillus plantarum, Lactobacillus jenseni, Lactobacillus casei, Lactobacillus fermentum, Lactococcus lactis, Pedioccocus acidilacti, Pedioccocus pentosaceus, Pedioccocus urinae, Leuconostoc mesenteroides, Bacillus coagulans, Bacillus subtilis, Bacillus laterosporus, Bacillus laevolacticus, Sporolactobacillus inulinus and mixtures thereof. Breath fresheners are also known by the following trade names: Retsyn™, Actizol™ and Nutrazin™. Examples of malodor-controlling compositions are also included in U.S. Pat. No. 5,300,305 to Stapler et al. and in U.S. Patent Application Publication Nos. 2003/0215417 and 2004/0081713 which are incorporated in their entirety herein by reference for all purposes.

### Dental Care Ingredients

Dental care ingredients (also known as oral care ingredients) may include but are not limited to tooth whiteners, stain removers, oral cleaning, bleaching agents, desensitizing agents, dental remineralization agents, antibacterial agents, anticaries agents, plaque acid buffering agents, surfactants and anticalculus agents. Non-limiting examples of such ingredients can include, hydrolytic agents including proteolytic enzymes, abrasives such as hydrated silica, calcium carbonate, sodium bicarbonate and alumina, other active stain-removing components such as surface-active agents, including, but not limited to anionic surfactants such as sodium stearate, sodium palminate, sulfated butyl oleate, sodium oleate, salts of fumaric acid, glycerol, hydroxylated lecithin, sodium lauryl sulfate and chelators such as polyphosphates, which are typically employed as tartar control ingredients. In some embodiments, dental care ingredients can also include tetrasodium pyrophosphate and sodium tri-polyphosphate, sodium bicarbonate, sodium acid pyrophosphate, sodium tripolyphosphate, xylitol, sodium hexametaphosphate, theobromine, and the like.

In some embodiments, peroxides such as carbamide peroxide, calcium peroxide, magnesium peroxide, sodium peroxide, hydrogen peroxide, and peroxydiphospate are included. In some embodiments, potassium nitrate and potassium citrate are included. Other examples can include casein glycomacropeptide, calcium casein peptone-calcium phosphate, casein phosphopeptides, casein phosphopeptide-amorphous calcium phosphate (CPP-ACP), and amorphous calcium phosphate. Still other examples can include papaine, krillase, pepsin, trypsin, lysozyme, dextranase, mutanase, glycoamylase, amylase, glucose oxidase, and combinations thereof.

Further examples can include surfactants such as sodium stearate, sodium ricinoleate, and sodium lauryl sulfate surfactants for use in some embodiments to achieve increased prophylactic action and to render the dental care ingredients more cosmetically acceptable. Surfactants can preferably be detersive materials which impart to the composition detersive and foaming properties. Suitable examples of surfactants are polysorbate 80, poloxamer, water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

In addition to surfactants, dental care ingredients can include antibacterial agents such as, but not limited to, triclosan, chlorhexidine, zinc citrate, silver nitrate, copper, limonene, and cetyl pyridinium chloride. In some embodiments additional anticaries agents can include fluoride ions or fluorine-providing components such as inorganic fluoride salts. In some embodiments, soluble alkali metal salts, for example, sodium fluoride, potassium fluoride, sodium fluorosilicate, ammonium fluorosilicate, sodium monofluorophosphate, as well as tin fluorides, such as stannous fluoride and stannous chloride can be included. In some embodiments, a fluorine-containing compound having a beneficial effect on the care and hygiene of the oral cavity, e.g., diminution of enamel solubility in acid and protection of the teeth against decay may also be included as an ingredient. Examples thereof include sodium fluoride, stannous fluoride, potassium fluoride, potassium stannous fluoride (SnF.sub.2-KF), sodium hexafluorostannate, stannous chlorofluoride, sodium fluorozirconate, and sodium monofluorophosphate. In some embodiments, urea is included.

Further examples are included in the following U.S. patents and U.S. published patent applications, the contents of all of which are incorporated in their entirety herein by reference for all purposes: U.S. Pat. No. 5,227,154 to Reynolds, U.S. Pat. No. 5,378,131 to Greenberg, U.S. Pat. No. 6,846,500 to Luo et al., U.S. Pat. No. 6,733,818 to Luo et al., U.S. Pat. No. 6,696,044 to Luo et al., U.S. Pat. No. 6,685,916 to Holme et al., U.S. Pat. No. 6,485,739 to Luo et al., U.S. Pat. No. 6,479,071 to Holme et al., U.S. Pat. No. 6,471,945 to Luo et al., U.S. Patent Publication Nos. 20050025721 to Holme et al., 2005008732 to Gebreselassie et al., and 20040136928 to Holme et al.

### Active Ingredients

Actives generally refer to those ingredients that are included in a the fondant layer and/or chewing gum region for the desired end benefit they provide to the user. In some embodiments, actives can include medicaments, nutrients, nutraceuticals, herbals, nutritional supplements, pharmaceuticals, drugs, and the like and combinations thereof.

Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, which is currently marketed as Viagra™, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of active ingredients contemplated for use in the present invention can include antacids, H2-antagonists, and analgesics. For example, antacid dosages can be prepared using the ingredients calcium carbonate alone or in combination with magnesium hydroxide, and/or aluminum hydroxide. Moreover, antacids can be used in combination with H2-antagonists.

Analgesics include opiates and opiate derivatives, such as Oxycontin™, ibuprofen, aspirin, acetaminophen, and combinations thereof that may optionally include caffeine.

Other drug active ingredients for use in embodiments can include anti-diarrheals such as Immodium™ AD, anti-histamines, anti-tussives, decongestants, vitamins, and breath fresheners. Also contemplated for use herein are anxiolytics such as Xanax™; anti-psychotics such as Clozaril™. and Haldol™; non-steroidal anti-inflammatories (NSAID's) such as ibuprofen, naproxen sodium, Voltaren™. and Lodine™, anti-histamines such as Claritin™, Hismanal™, Relafen™, and Tavist™; anti-emetics such as Kytril™ and Cesamet™; bronchodilators such as Bentolin™, Proventil™; anti-depressants such as Prozacrm, Zoloft™, and Paxil™; anti-migraines such as Imigra™, ACE-inhibitors such as Vasotec™, Capoten™ and Zestri™.; anti-Alzheimer's agents, such as Nicergoline™; and CaH-antagonists such as Procardia™, Adalat™, and Calan™, and the like.

The multi-layered chewing gum of the present invention can be used as a delivery system for probiotics. Probiotics are living microbial food additives, that have beneficial effect on the microbiological balance of the intestine and human health. Probiotics are used as functional food. Functional food is foodstuff, consumed additionally to usual food and containing bio-preparations (incl. probiotics) or other components favourably influencing human health or decreasing disease risks. Probiotics are prepared as components of food (probiotic yoghurt or cheese) or non-food preparations (lyophilised microbial cultures).

Most of probiotics are lactic acid bacteria, mainly lactobacilli. Lactobacilli are non-pathogenic micro-organisms, colonising the human intestinal and urogenital tract from early childhood to old age. Nowadays, several commercial probiotic lactobacilli are successfully used, among which Lactobacillus rhamnosus GG is the best known. Recently some new strains of lactobacilli have been described and patented, for example, L. reuterii isolated from animal organism and for this reason inappropriate for human usage.

Several strains of Lactobacillus fermentum are used for correction and stabilisation of intestinal micro-flora in case of dysbacterioses and urogenital infections with different ethiologies. The strain of micro-organism Lactobacillus fermentum 39 is used for producing the bacterial biological preparation (PCT/SU89/00264 (WO 91/05852), C12N 1/20, A61K 35/74, University of Tartu, 1991). The strain Lactobacillus fermentum 90-TS-4 (RU2133272, C12N 1/20, A61K 35/74, Akivo Lentsner et al., 1999) is characterised by lectin typing as a mannose-sensitive profile of the cell wall. The preparation is prescribed for use in gynaecology.

The prebiotics can be present in either the fondant layer or in the gum region layer of the multi-layered chewing gum of the present invention. If present in the chewing gum region layer, it is preferred that it is encapsulated or completely enclosed or encoded within the layer such as being surrounded by a shell, or other enclosures. Alternatively, the prebiotio can be present in the fondant layer. It may be enclosed within the fondant layer or present as a powder.

The popular H2-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients can include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, phosphatidylserine, tartaric acids and salts.

A variety of nutritional supplements may also be used as active ingredients including virtually any vitamin or mineral. For example, vitamin A, vitamin C, vitamin D, vitamin E, vitamin K, including vitamin K₂, vitamin B₆, vitamin B₁₂, thiamine, riboflavin, biotin, folic acid, niacin, pantothenic acid, sodium, potassium, calcium, magnesium, phosphorus, sulfur, chlorine, iron, copper, iodine, zinc, selenium, manganese, choline, chromium, molybdenum, fluorine, cobalt and combinations thereof, may be used.

Examples of nutritional supplements that can be used as active ingredients are set forth in U.S. Patent Application Publication Nos. 2003/0157213 A1, 2003/0206993 and 2003/0099741 A1 which are incorporated in their entirety herein by reference for all purposes.

Various herbals may also be used as active ingredients such as those with various medicinal or dietary supplement properties. Herbals are generally aromatic plants or plant parts and or extracts thereof that can be used medicinally or for flavoring. Suitable herbals can be used singly or in various mixtures. Active ingredients derived from herbal or botanical sources are sometimes referred to as phytochemicals. Classes of phytochemicals include, but are not limited to, polyphenols, flavonoids, flavanols, flavonols, flavanones, isoflavanones, anthocyanins, catechins, and flavonones. Commonly used herbs include Echinacea, Goldenseal, Calendula, Rosemary, Thyme, Kava Kava, Aloe, Blood Root, Grapefruit Seed Extract, Black Cohosh, Ginseng, Guarana, Cranberry, Gingko Biloba, St. John's Wort, Evening Primrose Oil, Yohimbe Bark, Green Tea, Ma Huang, Maca, Bilberry, Lutein, and combinations thereof.

Other non-herbal extracts can be used as active ingredients, for other purposes, e.g., flavoring. Examples include cocoa extracts, pomegranate extracts and the like.

### Effervescing System Ingredients

An effervescent system may include one or more edible acids and one or more edible alkaline materials. The edible acid(s) and the edible alkaline material(s) may react together to generate effervescence.

In some embodiments, the alkaline material(s) may be selected from, but is not limited to, alkali metal carbonates, alkali metal bicarbonates, alkaline earth metal carbonates, alkaline earth metal bicarbonates, and combinations thereof. The edible acid(s) may be selected from, but is not limited to, citric acid, phosphoric acid, tartaric acid, malic acid, ascorbic acid, and combinations thereof. In some embodiments, an effervescing system may include one or more other ingredients such as, for example, carbon dioxide, oral care ingredients, flavorants, etc.

For examples of use of an effervescing system in a chewing gum, refer to U.S. Provisional Patent No. 60/618,222 filed Oct. 13, 2004, and entitled “Effervescent Pressed Gum Tablet Compositions,” the contents of which are incorporated herein by reference for all purposes. Other examples can be found in U.S. Pat. No. 6,235,318, the contents of which are incorporated herein by reference for all purposes.

### Appetite Suppressor Ingredients

Appetite suppressors can be ingredients such as fiber and protein that function to depress the desire to consume food. Appetite suppressors can also include benzphetamine, diethylpropion, mazindol, phendimetrazine, phentermine, hoodia (P57), Olibra™, ephedra, chromium piccolinate, caffeine and combinations thereof. Appetite suppressors are also known by the following trade names: Adipex, Adipost™, Bontril™ PDM, Bontril™ Slow Release, Didrex™, Fastin™, Ionamin™, Mazanor™, Melfiat™, Obenix™, Phendiet™, Phendiet-105™, Phentercot™, Phentride™, Plegine™, Prelu-2™, Pro-Fast™, PT 105™, Sanorex™, Tenuate™, SanorexT™, Tenuate™, Tenuate Dospan™, Tepanil Ten-Tab™, Teramine™, and Zantry™. These and other suitable appetite suppressors are further described in the following U.S. patents, all of which are incorporated in their entirety by reference hereto: U.S. Pat. No. 6,838,431 to Portman, U.S. Pat. No. 6,716,815 to Portman, U.S. Pat. No. 6,558,690 to Portman, U.S. Pat. No. 6,468,962 to Portman, U.S. Pat. No. 6,436,899 to Portman.

### Potentiator Ingredients

Potentiators can consist of materials that may intensify, supplement, modify or enhance the taste and/or aroma perception of an original material without introducing a characteristic taste and/or aroma perception of their own. In some embodiments, potentiators designed to intensify, supplement, modify, or enhance the perception of flavor, sweetness, tartness, umami, kokumi, saltiness and combinations thereof can be included.

In some embodiments, examples of suitable potentiators, also known as taste potentiators include, but are not limited to, neohesperidin dihydrochalcone, chlorogenic acid, alapyridaine, cynarin, miraculin, glupyridaine, pyridinium-betain compounds, glutamates, such as monosodium glutamate and monopotassium glutamate, neotatne, thaumatin, tagatose, trehalose, salts, such as sodium chloride, monoammonium glycyrrhizinate, vanilla extract (in ethyl alcohol), sugar acids, potassium chloride, sodium acid sulfate, hydrolyzed vegetable proteins, hydrolyzed animal proteins, yeast extracts, adenosine monophosphate (AMP), glutathione, nucleotides, such as inosine monophosphate, disodium inosinate, xanthosine monophosphate, guanylate monophosphate, alapyridaine (N-(1-carboxyethyl)-6-(hydroxymethyl)pyridinium-3-ol inner salt, sugar beet extract (alcoholic extract), sugarcane leaf essence (alcoholic extract), curculin, strogin, mabinlin, gymnemic acid, 3-hydrobenzoic acid, 2,4-dihydrobenzoic acid, citrus aurantium, vanilla oleoresin, sugarcane leaf essence, maltol, ethyl maltol, vanillin, licorice glycyrrhizinates, compounds that respond to G-protein coupled receptors (T2Rs and T1Rs) and taste potentiator compositions that impart kokumi, as disclosed in U.S. Pat. No. 5,679,397 to Kuroda et al., which is incorporated in its entirety herein by reference. “Kokumi” refers to materials that impart “mouthfulness” and “good body”.

Sweetener potentiators, which are a type of taste potentiator, enhance the taste of sweetness. In some embodiments, exemplary sweetener potentiators include, but are not limited to, monoammonium glycyrrhizinate, licorice glycyrrhizinates, citrus aurantium, alapyridaine, alapyridaine N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, tagatose, trehalose, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and T1Rs) and combinations thereof.

Additional examples of potentiators for the enhancement of salt taste include acidic peptides, such as those disclosed in U.S. Pat. No. 6,974,597, herein incorporated by reference. Acidic peptides include peptides having a larger number of acidic amino acids, such as aspartic acid and glutamic acid, than basic amino acids, such as lysine, arginine and histidine. The acidic peptides are obtained by peptide synthesis or by subjecting proteins to hydrolysis using endopeptidase, and if necessary, to deamidation. Suitable proteins for use in the production of the acidic peptides or the peptides obtained by subjecting a protein to hydrolysis and deamidation include plant proteins, (e.g. wheat gluten, corn protein (e.g., zein and gluten meal), soybean protein isolate), animal proteins. (e.g., milk proteins such as milk casein and milk whey protein, muscle proteins such as meat protein and fish meat protein, egg white protein and collagen), and microbial proteins (e.g., microbial cell protein and polypeptides produced by microorganisms).

The sensation of warming or cooling effects may also be prolonged with the use of a hydrophobic sweetener as described in U.S. Patent Application Publication 2003/0072842 A1 which is incorporated in its entirety herein by reference. For example, such hydrophobic sweeteners include those of the formulae I-XI as set forth below:

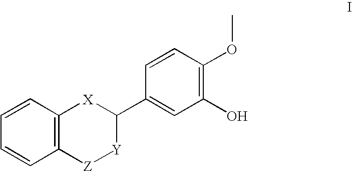

wherein X, Y and Z are selected from the group consisting of CH₂, O and S;

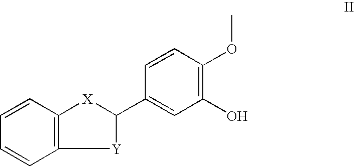

wherein X and Y are selected from the group consisting of S and O;

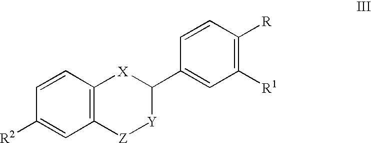

wherein X is S or O; Y is O or CH₂; Z is CH₂, SO₂ or S; R is OCH₃, OH or H; R¹ is SH or OH and R² is H or OH;

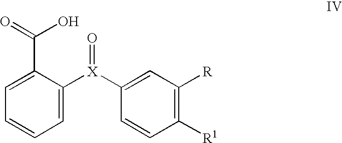

wherein X is C or S; R is OH or H and R¹ is OCH₃, or OH;

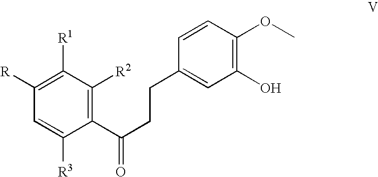

wherein R, R² and R³ are OH or H and R¹ is H or COOH;

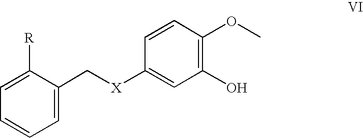

wherein X is O or CH₂, and R is COOH or H;

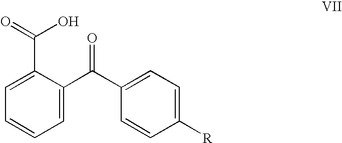

wherein R is CH₃CH₂, OH, N(CH₃)₂ or Cl;

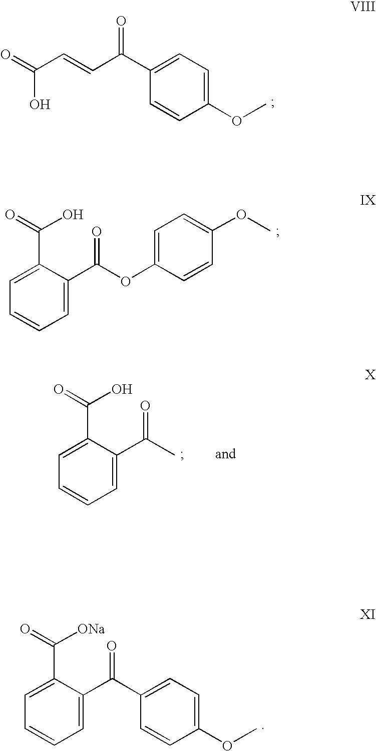

Perillartine may also be added as described in U.S. Pat. No. 6,159,509 also incorporated in its entirety herein by reference.

### Food Acid Ingredients

Acids can include, but are not limited to acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof.

### Micronutrient Ingredients

Micronutrients can include materials that have an impact on the nutritional well being of an organism even though the quantity required by the organism to have the desired effect is small relative to macronutrients such as protein, carbohydrate, and fat. Micronutrients can include, but are not limited to vitamins, minerals, enzymes, phytochemicals, antioxidants, and combinations thereof.

In some embodiments, vitamins can include fat soluble vitamins such as vitamin A, vitamin D, vitamin E, and vitamin K and combinations thereof. In some embodiments, vitamins can include water soluble vitamins such as vitamin C (ascorbic acid), the B vitamins (thiamine or B₁, riboflavin or B₂, niacin or B₃, pyridoxine or B₆, folic acid or B₉, cyanocobalimin or B₁₂, pantothenic acid, biotin), and combinations thereof.

In some embodiments minerals can include but are not limited to sodium, magnesium, chromium, iodine, iron, manganese, calcium, copper, fluoride, potassium, phosphorous, molybdenum, selenium, zinc, and combinations thereof.

In some embodiments micronutrients can include but are not limited to L-carnitine, choline, coenzyme Q10, alpha-lipoic acid, omega-3-fatty acids, pepsin, phytase, trypsin, lipases, proteases, cellulases, and combinations thereof.

Antioxidants can include materials that scavenge free radicals. In some embodiments, antioxidants can include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and combinations thereof.

In some embodiments phytochemicals can include but are not limited to cartotenoids, chlorophyll, chlorophyllin, fiber, flavanoids, anthocyanins, cyaniding, delphinidin, malvidin, pelargonidin, peonidin, petunidin, flavanols, catechin, epicatechin, epigallocatechin, epigallocatechingallate, theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof.

### Mouth Moistening Ingredients

Mouth moisteners can include, but are not limited to, saliva stimulators such as acids and salts and combinations thereof. In some embodiments, acids can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof.

Mouth moisteners can also include hydrocolloid materials that hydrate and may adhere to oral surface to provide a sensation of mouth moistening. Hydrocolloid materials can include naturally occurring materials such as plant exudates, seed gums, and seaweed extracts or they can be chemically modified materials such as cellulose, starch, or natural gum derivatives. In some embodiments, hydrocolloid materials can include pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, bacterial gums, and combinations thereof. Additionally, in some embodiments modified natural gums such as propylene glycol alginate, carboxymethyl locust bean gum, low methoxyl pectin, and their combinations can be included. In some embodiments, modified celluloses can be included such as microcrystalline cellulose, carboxymethylcellulose (CMC), methylcellulose (MC), hydroxypropylmethylcellulose (HPCM), and hydroxypropylcellulose (MPC), and combinations thereof.

Similarly, humectants which can provide a perception of mouth hydration can be included. Such humectants can include, but are not limited to glycerol, sorbitol, polyethylene glycol, erythritol, and xylitol. Additionally, in some embodiments, fats can provide a perception of mouth moistening. Such fats can include medium chain triglycerides, vegetable oils, fish oils, mineral oils, and combinations thereof.

### Throat Care Ingredients

Throat soothing ingredients can include analgesics, anesthetics, demulcents, antiseptic, and combinations thereof. In some embodiments, analgesics/anesthetics can include menthol, phenol, hexylresorcinol, benzocaine, dyclonine hydrochloride, benzyl alcohol, salicyl alcohol, and combinations thereof. In some embodiments, demulcents can include but are not limited to slippery elm bark, pectin, gelatin, and combinations thereof. In some embodiments, antiseptic ingredients can include domiphen bromide, dequalinium chloride, and combinations thereof.

In some embodiments, antitussive ingredients such as chlophedianol hydrochloride, codeine, codeine phosphate, codeine sulfate, dextromethorphan, dextromethorphan hydrobromide, diphenhydramine citrate, and diphenhydramine hydrochloride, and combinations thereof can be included.

In some embodiments, throat soothing agents such as honey, propolis, aloe vera, glycerine, menthol and combinations thereof can be included. In still other embodiments, cough suppressants can be included. Such cough suppressants can fall into two groups: those that alter the consistency or production of phlegm such as mucolytics and expectorants; and those that suppress the coughing reflex such as codeine (narcotic cough suppressants), antihistamines, dextromethorphan and isoproterenol (non-narcotic cough suppressants). In some embodiments, ingredients from either or both groups can be included.

In still other embodiments, antitussives can include, but are not limited to, the group consisting of codeine, dextromethorphan, dextrorphan, diphenhydramine, hydrocodone, noscapine, oxycodone, pentoxyverine and combinations thereof. In some embodiments, antihistamines can include, but are not limited to, acrivastine, azatadine, brompheniramine, chlorpheniramine, clemastine, cyproheptadine, dexbrompheniramine, dimenhydrinate, diphenhydramine, doxylamine, hydroxyzine, meclizine, phenindamine, phenyltoloxamine, promethazine, pyrilamine, tripelennamine, triprolidine and combinations thereof. In some embodiments, non-sedating antihistamines can include, but are not limited to, astemizole, cetirizine, ebastine, fexofenadine, loratidine, terfenadine, and combinations thereof.

In some embodiments, expectorants can include, but are not limited to, ammonium chloride, guaifenesin, ipecac fluid extract, potassium iodide and combinations thereof. In some embodiments, mucolytics can include, but are not limited to, acetylcysteine, ambroxol, bromhexine and combinations thereof. In some embodiments, analgesic, antipyretic and anti-inflammatory agents can include, but are not limited to, acetaminophen, aspirin, diclofenac, diflunisal, etodolac, fenoprofen, flurbiprofen, ibuprofen, ketoprofen, ketorolac, nabumetone, naproxen, piroxicam, caffeine and mixtures thereof. In some embodiments, local anesthetics can include, but are not limited to, lidocaine, benzocaine, phenol, dyclonine, benzonotate and mixtures thereof.

In some embodiments nasal decongestants and ingredients that provide the perception of nasal clearing can be included. In some embodiments, nasal decongestants can include but are not limited to phenylpropanolamine, pseudoephedrine, ephedrine, phenylephrine, oxymetazoline, and combinations thereof. In some embodiments ingredients that provide a perception of nasal clearing can include but are not limited to menthol, camphor, borneol, ephedrine, eucalyptus oil, peppermint oil, methyl salicylate, bornyl acetate, lavender oil, wasabi extracts, horseradish extracts, and combinations thereof. In some embodiments, a perception of nasal clearing can be provided by odoriferous essential oils, extracts from woods, gums, flowers and other botanicals, resins, animal secretions, and synthetic aromatic materials.

The multi-layered chewing gum of the present invention is prepared by art-recognized techniques. Each composition for each layer is prepared separately, i.e, the fondant layer is prepared separately from the composition of the chewing gum region layers and the composition of each chewing gum region layer is prepared separately. However, if any two compositions are identical, then obviously they are prepared together. Once the various composition for each layer are prepared, they are heated until the composition of each layer is malleable, and then the composition of each layer is extruded and layered, as will be described hereinbelow in more detail.

The chewing gum region layer is prepared by techniques known in the art. The chewing gum base is heated until it attains a flexible state and the other ingredients present in the chewing gum region layer, such as flavoring agent, sweetener, emulsifier, and the like are added to and mixed with the malleable gum base until a homogenous blend is obtained.

The fondant is prepared by art-recognized techniques. The base fondant is prepared by thoroughly mixing water, sweetener, bulking agent and stabilizer, e.g., TIC 15<CMC, at elevated temperatures above the boiling point of water, for example, to produce a homogenous mixture with a pasty texture. The mixture is then cooled to temperatures below the boiling point of water, such as about 125 to about 185° F. and more preferably from about 135 to about 155° F. Other components therein, such as the sensate, flavor, tastant, or functional agent or combination thereof, and any other additive present in the final fondant composition can then be mixed in to create the desired end product.

The multi-layer chewing gum is prepared using an extruder that has been modified. The following illustrates the procedure for making a three-layered chewing gum but the process for making a higher layered chewing gum is basically the same. In this embodiment, the extruder has been modified so that there are at least three outlet ports or nozzles, one for each layer. The ports may be on top of each other or on the same horizontal line. These models are commercially available

The fondant composition is fed into the middle port inlet, and the two chewing gum layer compositions are fed into the other two inlet ports. The fondant and the chewing gum components that will comprise the chewing gum layer are maintained at the same temperatures but at high enough temperature so that the chewing gum composition maintains the proper plasticity, in a malleable state and the fondant maintains the proper consistency and pasty texture. The temperature of the extruder, especially at the inlet and outlet ports, preferably range from about 100° F. to about 150° F., and more preferably from about 110 F to about 130° F.

In a three layered gum confection, the layers are co-extruded so that the fondant layer is sandwiched between the two chewing gum layers. It must be realized, however, that although a three layered chewing gum is the preferred embodiment of the present invention, any number of inlet and outlet nozzles may be conceivably modified onto the known extruders, and therefore any number of multiple layers is also contemplated by the present invention. The layered gum is then cooled, for example, in cooling tunnels, cut into pieces, sized according to choice and packaged by standard procedure and machinery that is commercially available.

Optionally, the outer layers of the multilayer chewing gum may be sanded, i.e., embedding of solid crystals on the surface of the outer layers of the chewing gum. Sanding consists of making fine sugar or polyol crystals, which adhere to the surfaces of the chewing gum of the present invention by moistening them before hand using techniques known to one of ordinary skill in the art, such as applying dry steam of a hot solution of gum arabic or maltodextrin to the surface and then drying the surfaces.

More specifically, an adhesive, i.e., a wetting syrup, is applied to the surface by techniques known to one of ordinary skill in the art, such as by panning, spray coating or any other procedure known in the art. The wetting syrup is preferably a tackifying syrup that is applied to the surface, e.g., a food acceptable tackifying solution. In a preferred embodiment, it includes gum arabic. In a sugar free gum, the wetting solution is a solution of a sugar alcohol and a tackifying agent such as gum arabic. The sugar alcohol is present in an amount ranging from about 40 to about 80 by weight of the wetting syrup and more preferably from about 60 to about 70% by weight of the wetting syrup. The tackifying agent is preferably present in about 1% to about 5% by weight of the wetting syrup. Still more preferably, the sugar alcohol is xylitol, mannitol, erythritol, maltitol, isomalt, and the like. Even still more preferably, the wetting solution sugar alcohol is maltitol. In that preferred embodiment, it is desirable that the maltitol be present in a concentration of about 66% by weight in the syrup of at least maltitol and gum arabic at about 1% to about 5% by weight of the syrup, the remainder being water. These percentages are by weight, based on the total weight of the wetting solution applied to the chewing gum pieces. Those skilled in the art appreciate that usage rate is indicative of the amount of gum arabic, the tackifying agent, that adheres to the chewing gum composition, rather than the amount actually present in the syrup processed.

A sugar alcohol is utilized in a preferred embodiment of the wetting syrup in order to further enhance the immediate burst of flavor and cooling provided by the sugar alcohol sanded particles. Maltitol is particularly preferred in this application because of its sweetness.

Once the solid chewing gum is wetted with the wetting syrup, the wetted chewing gum composition is next sanded with dry crystals of sugar or of a sugar alcohol having a negative heat of solution or particles of flavorant, sensate, or tastant utilizing sanding techniques known in the art. The sanding application occurs by applying the sugar or sugar alcohol particles or particles of the flavorant, sensate, or tastant onto the wetted surface of the chewing gum pieces by panning or by spray or any other method known in the art for sanding. The preferred process of the present invention for applying the wetting syrup to the surface chewing gum surface and for sanding is coating, e.g., by panning.

The crystals are thereupon applied to the wetting syrup-coated chewing gum composition solid pieces. If a polyol is used, a sufficient amount of polyol is present on the wetting syrup coated chewing gum to impart a coolness sensation when bit into by a consumer, such as humans. The crystals are applied such that the chewing gum pieces preferably include between about 5% and about 12% sanded crystals. More preferably, the crystals are present in a concentration of between about 5% and about 10%. Still more preferably, the sugar alcohol sanded crystals constitute between about 6% and about 9%. These percentages are by weight based on the total weight of the chewing gum sanded pieces.

If the chewing gum is a sugar containing chewing gum, it may be sanded with sugar, e.g., sucrose, glucose, polydextrose and the like or polyol as described hereinabove. If it is a sugarless chewing gum, solid sugars may not be used but polyols may be used instead. The process of sanding is described in copending application entitled “Sugar Alcohol Sanded Chewing Gum and Process For Making Same” having U.S. Ser. No. 11/432,053, the contents of which are incorporated by reference.

Alternatively, the multi-layered chewing gum may optionally be coated. The coating may comprise one or more ingredients that are typically found in coatings of chewing gums, such as magnesium stearate, cornstarch, sugars, polyols, cellulose ethers, such as hydroxypropylmethyl cellulose, e.g., film coating, acrylic polymers and copolymers, sugarless/sugarfree coatings or waxes. The coating can be provided in any known manner within the art of coating chewing gums. The coating may serve to protect the chewing gum composition core and retain moisture in the chewing gum during storage. Alternatively, the coating may contain a flavorant, tastant, sensant or a functional agent, as in the gum.

Thus, whether the chewing gum is sanded or has a coating, as described hereinabove, this provides another opportunity to provide another area what can provide a different or complementary taste, flavor, sensate or functional agent to that which is present in the gum. For example, the coating or sanded material may contain xylitol which provides the initial coolness sensation.

The chewing gum composition is quite different and is unique. As indicated herein, in the chewing gum composition, the fondant and the chewing gum region are present in a separate and distinct layers and the contents do not mix.

The present composition has a higher moisture content than that normally found in chewing gums. Although the chewing gum region of the product may have low water activity, the fondant is hydrophilic and has a higher water activity than most other gum products. Thus, the product of the present invention has more moisture than other chewing gums and therefore does not dry out as quickly when chewing as other chewing gums. Moreover, it is a juicier product. Further, by maintaining separate layers for the gum region and the fondant, there is no migration of water, flavors, tastants, colorants and the like between the various layers so that no barrier layer is used to separate the various layers of the gum. That is to say, there is no migration of ingredients between layers. This is contrast to center-filled compositions, in which there is a tendency for moisture, taste or color migration from the center or for flavor leaking out. Further, center-filled compositions can only contain a certain amount of material in the center, while the chewing gum of the present invention has a capacity of holding much more material in the center regions. Further, the process for preparing the present invention is quite simple. Other than the extruder, there is no special apparatus required to make the product.

Moreover, by making the layers co-extensive, the middle layers have a large volume of sensate, functional agent, flavorant or tastant. Thus, the chewing gum composition of the present invention can hold greater amounts of the aforementioned components, which tends to intensify the taste, sensate or flavor or potentially administer more functional agent to the consumer.

Unless indicated to the contrary, the percentages and ratios are by weight of the product, not dry weight.

Unless indicated to the contrary, the singular denotes the plural and vice versa.

The following example further illustrates the present invention.

### EXAMPLE

### Preparation of a Three-Layered Gum

In this example, the two gum chewing layers are comprised of the following components:

| Ingredients | % by weight |
|---|---|
| Bulk sweetener (such as polyol) | 59.74 |
| Gum base | 24.46 |
| Bulking agent | 11.51 |
| Lecithin | 0.35 |
| Color | 0.30 |
| Aspartame | 0.25 |
| Ace-K-Powder | 0.09 |
| Flavors | 3.30 |

A gum base, which has been melted at 180-200° F., color, and lecithin are added into a heated gum mixer set at 125° F.-130° F. The contents are mixed until homogenous. The bulking agent is next added to the homogenous mixture, and the contents are again thoroughly mixed until homogenous. The bulk sweetener (e.g. polyol) is added thereto and the contents are mixed. Thirty seconds alter adding the bulk sweetener, liquid flavor is added, and the contents are mixed until homogenous. The remaining flavor and the intense sweeteners, Aspartame and Ace-K-Powder, are added, and the contents are thoroughly mixed until a homogenous blend is formed. The temperature of the gum is 115-125° F. After dusting the mixer with talc to prevent sticking of the gum to the mixer, the finished gum composition is removed from the mixer.

The base fondant in this example is comprised of the following composition

| Ingredient | % |
|---|---|
| Xylitol | 70.0 |
| Lycasin (85% dry base) | 20.4 |
| TIC 15 CMC | 1.0 |
| Water | 8.6 |

The Lycasin and water are added to a vessel that is preheated to 220° F. The xylitol and TIC 15 CMC are added to the Lycasin and water. The resulting mixture is passed through a heat exchanger so that the final temperature of 270° F. is reached and maintained. After the mixture is homogenous, the mixture is passed to a cooling wheel to adjust the temperature to about 145° F. The mixture is then passed through a fondant beater and collected in containers.

The fondant so prepared is cut into smaller pieces and added to a steam-heated gum base melter. The steam, however, is shut off when the fondant is added. While adding the fondant to the melter, it is intermittently mixed. Once all the fondant has been added, the contents are mixed until the temperature of fondant is about 115° F. or pasty in texture. Color, flavor, and functional ingredients (e.g., tastants, sensates, flavorants and/or functional agents) are added to the pasty mixture and the fondant mixture is continued to be heated and mixed until its temperature is about 125° F. Small quantities of the fondant are transferred into panned sprayed containers, and added to the extruder.

The gum and the fondant are passed through a rope extruder, with the fondant being fed into the center hopper and the gum fed into the right and left hoppers.

The extruder barrel and head temperature are set at 120° F.

The fondant and gum are co-extruded.

The gum and fondant layers are extruded into a rope form and are carried by conveyor belts through the cooling tunnel. Conditions used within the cooling tunnel are ˜35° F., and 40-60% R.H.

The ropes are dusted with a non-hydroscopic agent to prevent sticking to the equipment. Then the ropes are fed through a metal detector to the cut-and-wrap machine to produce the three-layered chewing gum in the approximately shaped cubes or logs.

The product is collected in shallow trays and cured for 12 hours at 55-75° F./45%-60% R.H.

The above preferred embodiments and examples are given to illustrate the scope and spirit of the present invention. The embodiments and examples described herein will make apparent to those skilled in the art other embodiments and examples. These other embodiments and examples are within the contemplation of the present invention. Therefore, the present invention should be limited only the appended claims.

What is claimed is:

1. A multi-layered chewing gum, comprising alternative layers of a fondant and a chewing gum region comprising a chewing gum composition, each layer being separate and discrete and co-extensive, said multilayered chewing gum comprised of at least three layers, wherein the outer layers are comprised of said chewing gum composition, and the fondant layer is sandwiched between the layers comprised of a chewing gum region, said fondant comprised of a first component selected from tastant, a flavor, a sensate and a functional agent and the gum region being comprised of a second component selected from tastant, flavor, sensate and a functionality agent, said first component being distinct from or the same as said second component.

2. The multi-layered chewing gum of claim 1 comprised of three layers where the outer layers are each comprised of a gum region and the middle layers is comprised of the fondant.

3. The multi-layered chewing gum of claim 1 or 2 wherein the fondant comprises polyol or a blend of polyols.

4. The multi-layered chewing gum of claim 1 or 2 wherein the fondant is comprised of a mixture of xylitol and malitol syrup.

5. The multi-layered chewing gum of claim 1 or 2, wherein the xylitol is present in amounts ranging from about 50% to about 90% by weight of the fondant.

6. The multi-layered chewing gum according to claim 5 wherein the xylitol ranges from about 60% to about 80% by weight.

7. The multi-layered chewing gum according to claim 1 wherein the water content of the fondant range from about 5 to about 10% by weight of the fondant.

8. The multi-layered chewing gum according to claim 1 or 2, wherein the fondant contains a first component which is selected from the group consisting of a tastant, a sensate and a flavor and each chewing gum layer contains a second component which is complementary thereto or which intensifies the taste, flavor or sensation of the first component.

9. The multi-layered chewing gum according to claim 1 or 2 containing one or more functional agents, wherein the functional agent is in the fondant layer, the gum region layer or in both the gum region layers and the fondant layers.

10. The multi-layered chewing gum according to claim 1 or 2, wherein the fondant layer is a chocolate fondant.

11. The multi-layered chewing gum according to claim 1 or 2, wherein the fondant layer is comprised of a teeth whitener.

12. The multi-layered chewing gum according to claim 1 or 2 where the outer surfaces are sanded with polyol.

13. The multi-layered chewing gum according to claim 1 or 2 which contains a coating on the outer surfaces of the gum.

14. The multi-layered chewing gum according to claim 1 or 2 which is a sugarless chewing gum.

15. The multi-layered chewing gum according to claim 1 or 2 wherein the chewing gum region of each layer contains a first tastant and the fondant contains a second tastant, which may be same or different.

16. The multi-layered chewing according to claim 1 or 2, wherein the chewing gum region of each layer contains a first sensate and the fondant contains a second sensate which may be the same or different from first sensate.

17. The multi-layered chewing gum according to claim 1 or 2, wherein the chewing gum region of each layer contains a first flavorant and the fondant layer contains a second flavorant which may be the same or different from the first flavorant.
